(19) United States
(12) Patent Application Publication
(10) Pub. No.: US 2012/0034706 A1
(43) Pub. Date: Feb. 9, 2012

(54) METHOD FOR ASSESSING INFLAMMATORY CONDITION

(75) Inventors: Yukitaka Ueki, Sasebo-shi (JP); Nobuhito Masuda, Tokyo (JP); Kyoko Miyamoto, Tokyo (JP); Chihiro Miyazawa, Tokyo (JP); Yukio Sudo, Tokyo (JP)

(73) Assignees: HAKUJYUJIKAI MEDICAL INC., Sasebo-shi (JP); Perseus Proteomics Inc., Meguro-ku (JP)

(21) Appl. No.: 12/964,077

(22) Filed: Dec. 9, 2010

(30) Foreign Application Priority Data
Aug. 6, 2010 (JP) 2010-176845

(51) Int. Cl. G01N 33/53

(52) U.S. Cl. 436/501

(57) ABSTRACT

Provided a method for correctly assessing an inflammatory condition of a patient who is receiving a therapy with an IL-6 inhibitor. The method for assessing an inflammatory condition of a patient who is receiving an IL-6 inhibitor, including determining a PTX3 level of a sample derived from a patient who is receiving an IL-6 inhibitor.

### BACKGROUND OF THE INVENTION

1. Field of the Invention

The present invention relates to a method for assessing an inflammatory condition of a patient who is receiving an IL-6 inhibitor.

2. Background Art

PTX3, which is also called pentraxin, pentaxin, TSG-14, or MPTX3, is a secretion protein belonging to the pentraxin family and is known to be expressed in human umbilical cord vessel endothelium cells stimulated with interleukin 1 (IL-1) (Non Patent Document 1).

The pentraxin family is generally divided into the group of long pentraxins and the group of short pentraxins. C-reactive protein (CRP) and serum amyloid P component (SAP), which are known as inflammation-related proteins, belong to the short pentraxin group and are similar to each other in terms of structure (Non Patent Document 2). Particularly, CRP is widely used as an inflammatory marker.

In patients of rheumatoid arthritis, not only variation in CRP level reflects the extent of inflammation attributed to rheumatoid arthritis, but also an increase in CRP level reflects an inflammatory condition attributed to other factors, such as an infection. In recent years, tocilizumab, which is an epochal remedy for rheumatoid arthritis or the like, has been available on the market. Tocilizumab is an anti-IL-6 receptor antibody which inhibits the action of IL-6. CRP is synthesized in the liver under positive control of IL-6 produced during inflammation and secreted into the blood. Therefore, in patients placed under administration of tocilizumab, the synthesis pathway of CRP is inhibited by tocilizumab, thereby failing to produce CRP. In this case, CRP cannot be employed as an inflammatory marker, which is problematic. Since tocilizumab has an action mechanism inhibiting IL-6, a symptom such as fever is masked, and an increase in blood CRP level cannot be observed. As a result, patients placed under administration of tocilizumab are readily infected, and the infection is readily aggravated. More seriously, the condition of such a patient becomes severe while the patient himself or herself or a relevant doctor fails to recognize that the patient has suffered from the infection. Thus, the inflammatory condition of a patient who is receiving tocilizumab must be assessed by a very small change in CRP level or leucocyte count or through very careful observation of the symptom. Under such circumstances, there is demand for an inflammatory marker which can be used also in patients who are receiving tocilizumab.

Meanwhile, it is known that PTX3, which has a structure similar to that of CRP, also serves as an inflammatory marker (Non Patent Document 3). It has been reported that, regarding rheumatoid arthritis, higher expression of PTX3 protein is observed in synovial fluid obtained from rheumatoid arthritis patients, as compared with a normal control, and that PTX3 protein is highly expressed in a synovial membrane tissue collected from rheumatoid arthritis patients (Non Patent Document 4).
Non Patent Document 1: Arthritis and Rheumatism, 44/12 (2841-50), 2001
Non Patent Document 2: Breviario et al.: J. Biol. Chem., 267(31), 22190-7 (1992)
Non Patent Document 3: Bottazzi et al.: J. Leucoc. Biol., 79(5), 909-12, (2006)
Non Patent Document 4: Luchetti et al.: Clin. Exp. Immunol., 119: 196-202 (2000)

### SUMMARY OF THE INVENTION

An object of the present invention is to provide a method for correctly assessing an inflammatory condition of a patient who is receiving a therapy with an IL-6 inhibitor.

In order to correctly assess an inflammatory condition of a patient who is receiving an IL-6 inhibitor, the present inventors have measured the PTX3 level of the patient in addition to the CRP level, and have investigated the relationship between the blood PTX3 and CRP levels and the inflammatory condition; for example, amelioration or aggravation in inflammation attributable to an inflammatory disease, occurrence of inflammation caused by infection, etc. As a result, the inventors have found that the PTX3 level remains at a low level in the case where inflammation has been ameliorated and was clearly elevated in the case of aggravation of inflammation or occurrence of infection, while the CRP level of the patient remains at a low level even during aggravation of inflammation or occurrence of infection, whereby the PTX3 level serves as a useful marker for correctly assessing a inflammatory condition in the patient who is receiving an IL-6 inhibitor. The present invention has been accomplished on the basis of this finding.

Accordingly, the present invention provides a method for assessing an inflammatory condition of a patient who is receiving an IL-6 inhibitor, the method including determining the PTX3 level of a sample derived from the patient who is receiving an IL-6 inhibitor.

Further, the present invention provides the aforementioned assessing method, in which the inflammatory condition may be an inflammatory condition attributed to a disease to which an IL-6 inhibitor is applied, or an inflammatory condition attributable to an infection.

Further, the present invention provides the aforementioned assessing method, in which assessment may be performed in combination of the aforementioned CRP level with the CRP level of a sample derived from the inflammatory disease patient who is receiving an IL-6 inhibitor.

According to the present invention, problems which have specifically arisen in patients placed under administration of an IL-6 inhibitor can be solved. That is, aggravation of inflammation such as fever and occurrence of an infection, which have not been detected, can be correctly detected. Thus, such patients can be treated through an appropriate therapy in addition to administration of an IL-6 inhibitor thereto.

### BRIEF DESCRIPTION OF THE DRAWINGS

FIG. 1 is a graph showing the change over time in average DAS28-ESR of 26 rheumatoid arthritis patients placed under a tocilizumab therapy;

FIG. 2 is a graph showing the changes over time in blood CRP level and blood PTX3 level of 26 rheumatoid arthritis patients placed under a tocilizumab therapy, the base line representing a CRP level before start of the therapy;

FIG. 3 is a graph showing the changes over time in DAS28-ESR, blood CRP level, and blood PTX3 level of a patient who has been infected during a tocilizumab therapy;

FIG. 4 is a graph showing the changes over time in DAS28-ESR, blood CRP level, and blood PTX3 level of a patient who has been infected during a tocilizumab therapy;

FIG. 5 is a graph showing the changes over time in DAS28-ESR, blood CRP level, and blood PTX3 level of a rheumatoid arthritis patient who has exhibited aggravated joint pain and swelling during a tocilizumab therapy;

FIG. 6 is a graph showing the changes over time in PTX3/CRP ratio of the patients in Example 3; and

FIG. 7 is a graph showing the change over time in PTX3/CRP ratio of the patient in Example 4.

### DETAILED DESCRIPTION OF PREFERRED EMBODIMENTS

The present invention is directed to assessment of an inflammatory condition of a patient who is receiving an IL-6 inhibitor. As used herein, the term “IL-6 inhibitor” refers to a drug which suppresses the biological activity of IL-6. No particular limitation is imposed on the IL-6 inhibitor, so long as it is a drug which suppresses the biological activity of IL-6. For example, the IL-6 inhibitor is a drug which suppresses the biological activity of IL-6, an IL-6 receptor or gp130 (gp130: a glycoprotein which has a molecular weight of about 130 kD and which associates with an IL-6 receptor to thereby transmit a signal into a cell). Specific examples of the IL-6 inhibitor include an anti-IL-6 antibody, an anti-IL-6 receptor antibody, an anti-gp130 antibody, and a peptide, a protein, or a low-molecular-weight compound which inhibits IL-6, an IL-6 receptor, or gp130. Examples of the anti-IL-6 antibody include CNTO328. Examples of the anti-IL-6 receptor antagonist include tocilizumab. Among them, an anti-IL-6 receptor antibody is preferred, with tocilizumab being particularly preferred. The aforementioned IL-6 inhibitor can be used in the treatment of a disease which involves an undesirable increase in IL-6 level. Examples of the disease or condition which can be treated with the IL-6 inhibitor include rheumatoid arthritis, juvenile idopathic arthritis, Castleman's disease, Crohn's disease, ulcerative colitis, cardiac myxoma, myeloma, adult Still's disease, polychondritis, Takayasu's arteritis, reactive arthritis, and RS3PE syndrome. The present invention can be applied not only to the diseases which can be treated with the IL-6 inhibitor, but also to a patient who is receiving an IL-6 inhibitor and particularly preferable to a patient suffering from rheumatoid arthritis. Among such patients who are receiving an IL-6 inhibitor, a patient who exhibits a CRP level lower than its threshold value for determining the inflammatory condition due to the effect of the IL-6 is preferable.

In the present invention, the PTX3 level of a sample derived from such a patient is determined. No particular limitation is imposed on the type of the sample, so long as the sample may contain PTX3 protein. Preferably, the sample is obtained from a human. Specific examples of the sample include blood, plasma, extravascular fluid, cerebrospinal fluid, synovial fluid, pleural fluid, serum, lymph, saliva, and urine. Of these, blood, serum, and plasma are preferred.

The PTX3 level may be determined through any known method per se. Among them, immunological assay employing an anti-PTX3 antibody is preferred. The anti-PTX3 antibody may be a polyclonal antibody or a monoclonal antibody.

Examples of the immunological assay employed in the present invention include radioimmunoassay, enzyme immunoassay, fluorescent immunoassay, luminescent immunoassay, immunoprecipitation, immunoturbidimetry, Western blotting, immunostaining, and immunodiffusion. Of these, enzyme immunoassay is preferred, with enzyme-linked immunosorbent assay (ELISA, e.g., sandwich ELISA) being particularly preferred. The aforementioned immunological assay methods including ELISA may be performed through a method known to those skilled in the art.

In one typical detection method employing an anti-PTX3 antibody an anti-PTX3 antibody is immobilized on a support, and a sample is added to the support, followed by incubating, whereby a PTX3 protein is linked to the PTX3 antibody. After that, the support is washed, and the PTX3 protein linked to the support via the anti-PTX3 antibody is detected, to thereby detect the PTX3 protein present in the sample. Alternatively, the immunoagglutination method is also preferred.

The PTX3 level of a sample derived from an inflammatory disease patient who is receiving an IL-6 inhibitor correctly reflects the inflammatory condition of the patient. In other words, in the case where the inflammation is suppressed through administration of the IL-6 inhibitor, the PTX3 level is low. When the condition (e.g., fever, pain, or swelling) is aggravated, the PTX3 level increases. When inflammation is caused by an infection, the PTX3 level also increases. In contrast, the CRP level, which is generally employed as an inflammatory marker, is lowered by administration of the IL-6 inhibitor. Even when the condition of the patient is aggravated, or the patient suffers from an infection, the CRP level remains low.

Thus, the PTX3 level of a patient of the aforementioned type correctly reflects the inflammatory condition. Therefore, when a target patient exhibits a PTX3 level higher than that of a healthy subject or that of an inflammatory disease patient whose inflammation is suppressed by an IL-6 inhibitor administered thereto, the target patient can be assessed to have inflammation. Whether or not the PTX3 level the target has increased can be determined on the basis of the presence of a statistical difference between the PTX3 level of the target and that obtained in advance from a healthy subject or that obtained in advance from a patient whose inflammation is suppressed by an IL-6 inhibitor administered thereto. In this case, the condition in which inflammation is observed includes the aforementioned aggravation of the inflammation and the presence of inflammation caused by an infection.

Moreover, the inflammatory condition of a patient who is receiving an IL-6 inhibitor can be assessed by observing the change over time of the PTX3 level of the patient. For example, when the PTX3 level of a patient at a certain timing is higher than the previously measured PTX3 level of the patient, the inflammation condition thereof can be assessed to be aggravated.

As described above, the CRP level of the patient is immediately lowered through administration of an IL-6 inhibitor thereto, and remains low even when the inflammation is caused in the patient thereafter. Thus, through combination of the PTX3 level and the CRP level, the inflammatory condition of a patient who is receiving an IL-6 inhibitor can also be assessed. Specifically, the inflammatory condition of the patient can be effectively assessed through measurement of the PTX3/CRP ratio. Since both the PTX-3 and the CRP level of a patient who is not receiving an IL-6 inhibitor are elevated by the presence of an inflammatory condition, the PTX3/CRP ratio does not drastically change depending on the degree of the inflammatory condition. In contrast, the PTX3/CRP ratio of a patient who is receiving an IL-6 inhibitor significantly increases by the presence of an inflammatory condition. When the PTX3 (ng/mL)/CRP (mg/dL) ratio of a patient who is receiving an IL-6 inhibitor is 100 or higher, the patient is suspected to have an inflammatory condition. The threshold of the PTX3/CRP ratio for determining the inflammatory condition is preferably set to 90 to 160, more preferably 95 to 140, and still more preferably 100 to 120. In the above case, the PTX3/CRP ratio is calculated by using a PTX3 level (ng/mL) and a CRP level (mg/dL). When a unit other than ng/mL or mg/dL is employed, the new unit may be converted to ng/mL or mg/dL. Notably, when the CRP level of a measurement system used is 0, the lowest detection value of the measurement system is employed as the CRP level, to thereby calculate the PTX3/CRP ratio.

Moreover, the inflammatory condition of a patient who is receiving an IL-6 inhibitor can be assessed by observing the change over time of the PTX3/CRP ratio of each of patients. For example, when the PTX3/CRP ratio of a patient at a certain timing is greater than the previously measured PTX3/CRP ratio of the patient, the inflammation condition thereof can be assessed to be aggravated.

The CRP level may be determined through a known method. Assay kits employing ELISA, immunoturbidimetry, the latex agglutination method, etc. are available on the market.

### EXAMPLES

### Example 1

### Target

Twenty-six rheumatoid arthritis patients were tested. To each patient, tocilizumab (8 mg/kg) was administered through intravenous dripping at intervals of 4 weeks. Biological data were collected from each patient every four weeks in a period to maximum of week 60. The characteristics of the patient group are shown in Table 1.

Before and immediately after administration of tocilizumab, the DAS28-ESR score, CRP level, and PTX3 level of each patient were measured. At the time of the first administration of tocilizumab, the first measurement was performed. Similarly, the second measurement was performed at the time of the second administration carried out four weeks after the first administration. The measurement was performed 15 times (i.e., to maximum of week 60). The serum CRP level was measured by means of LPIA-ACE CRP-LII (product of Mitsubishi Chemical Medience Corporation), and the plasma PTX3 level (of EDTA-added plasma) was measured by means of a PTX3-ELISA kit (product of Perseus Proteomics Inc.). When the patient was suspected to suffer from an infection, the white blood cell count (WBC) thereof was also measured.

TABLE 1
Total cases (n = 26)

| | |
|---|---|
| Age (year) | 56.9 ± 13.7 |
| Sex (female/male) | 23/3 |
| Disease duration (year) | 13.8 ± 10.7 |
| Stage (n) | I: 0, II: 3, III: 4, IV: 19 |
| Class (n) | 1: 0, 2: 23, 3: 3, 4: 0 |
| Tenderness/joint pain (/28 joints) | 15.2 ± 7.9 |
| Swelling/joint pain (/28 joints) | 4.8 ± 3.2 |
| General VAS (mm) | 48.0 ± 19.5 |
| ESR (mm/h) | 61.8 ± 35.9 |
| CRP (mg/dL) | 1.99 ± 2.19 |
| DAS28-ESR | 5.97 ± 1.41 |
| Combined with DMARDs (n) | 23 cases (88.5%) MTX: 12 (7.5 ± 1.2 |
| | mg/week) |
| | SASP: 1 LFM: 5 TAC: 3 |
| Combined with steroid (n) | 14 cases (53.8%) (6.3 ± 2.9 mg/day) |

The DAS28-ESR score was calculated from the following parameters.

(1) Joint with pain/tenderness (Ritchie's joint index) (T28)
(2) Number of swelled joints (S28)
(3) General health conditions of patient (VAS100 mm)
(4) Erythrocyte sedimentation rate (ESR), unit: mm/h

More specifically, the DAS28-ESR score was calculated by applying (1) to (4) above to the following formula.

DAS28-ESR=0.555×√(T28)+0.284×√(S28)+0.7×LN(ESR)+0.0142×(VAS)

Both (1) and (2) were evaluated at 28 joints. The specific calculation of the DAS28-ESR including the parameters obtained at 28 joints and the VAS evaluation may be performed through a method known in the art (see Prevoo M L L et al.: Modified disease activity scores that include twenty-eight-joint counts. Development and validation in a prospective longitudinal study of patients with rheumatoid arthritis. Arthritis Rheum. 38: 44-48, 1995).

### Example 2

### Overall Results

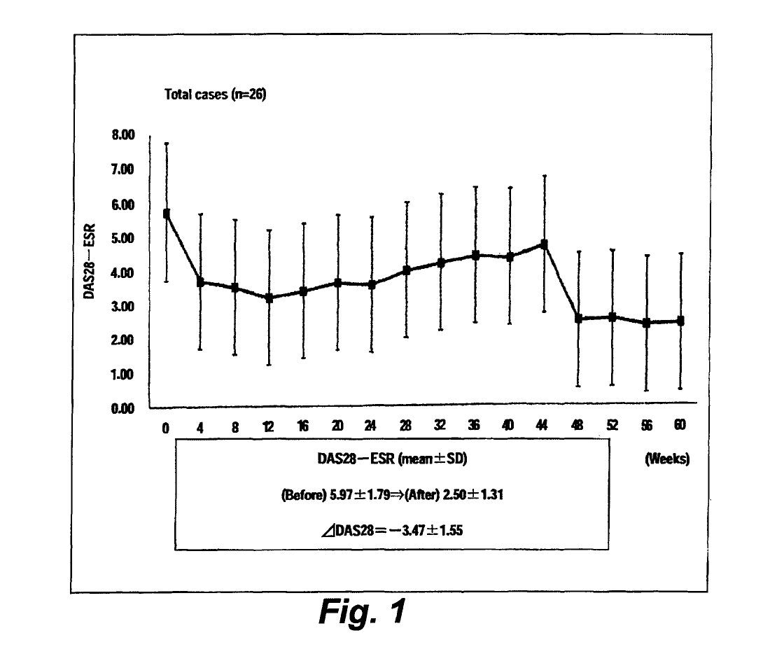

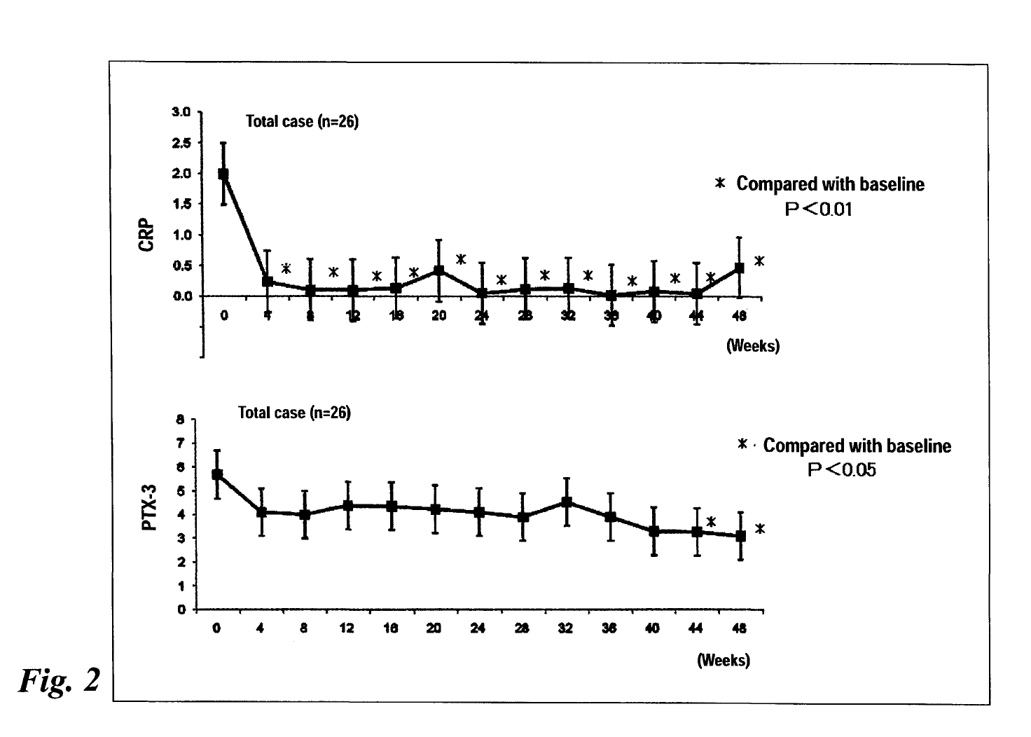

FIG. 1 shows the change over time in average DAS-ESR level (26 cases), and FIG. 2 shows the changes over time in average CRP level and that in average PTX3 level. The CRP level was significantly lowered at week 4 of administration of tocilizumab or later, as compared with the CRP level before the treatment. The PTX3 level was significantly lowered at week 44 of administration of tocilizumab or later, as compared with the PTX3 level before the treatment (FIG. 2).

### Example 3

### Infection and CRP Level or PTX3 Level

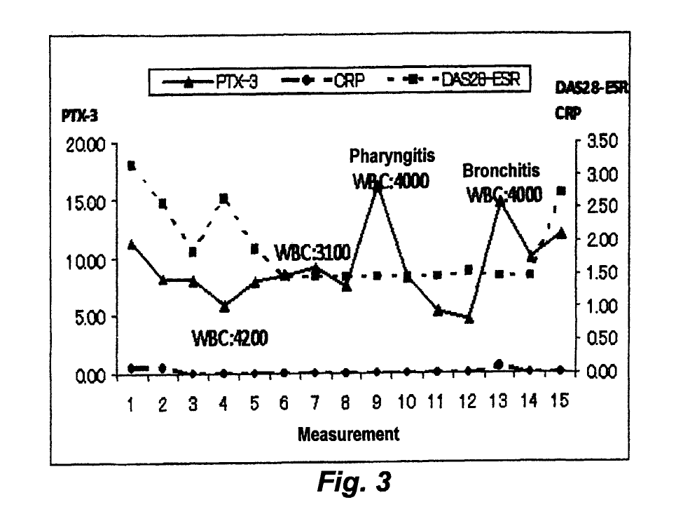

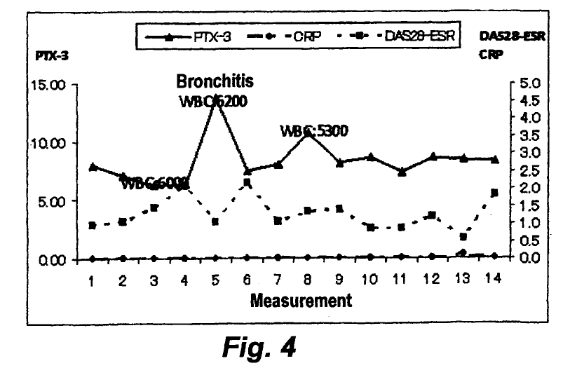

FIG. 3 (case 2) and FIG. 4 (case 28) show the change over time in DAS28-ESR, CRP level, and PTX3 level of each of two patients who have been infected during a tocilizumab therapy. The infection was diagnosed by a doctor based on the medical record, the chief complaint of the patients and observations such as symptoms, physical findings, and diagnostic imaging.

The CRP level of the patients, which is generally employed as an inflammatory marker, did not increase when the patients infected. Also, the white blood cell count (WBC, cells/μL) did not significantly increase. However, the PTX3 level was found to increase as the expression of clinically observed symptoms attributed to the infection.

### Example 4

### Aggravation of Joint Pain/Swelling and PTX3

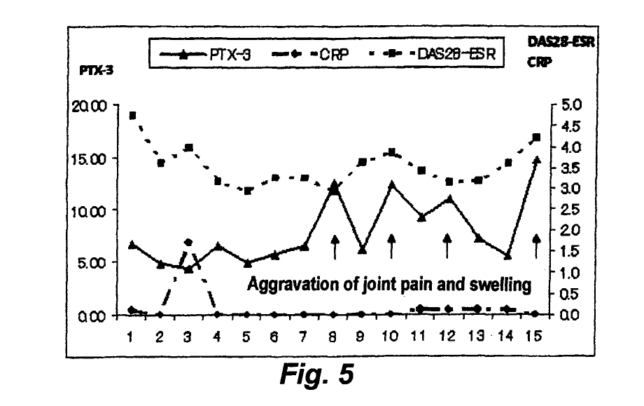

FIG. 5 (case 27) shows the changes over time in DAS28-ESR, CRP level, and PTX3 level of a patient who has exhibited aggravated joint pain and swelling during a tocilizumab therapy. The presence of joint pain and swelling was confirmed by a doctor on the basis of physical findings. While the CRP level and DAS28-ESR were virtually unvaried due to aggravation of joint pain and swelling, the PTX3 level was sharply elevated.

### Example 5

### PTX3/CRP Ratio and Inflammation

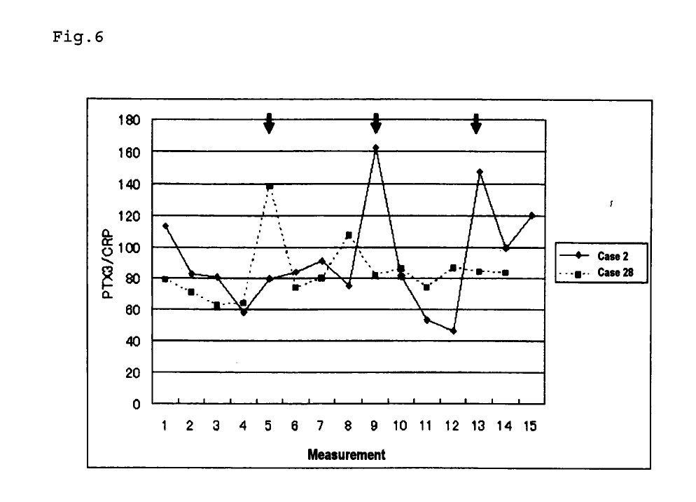

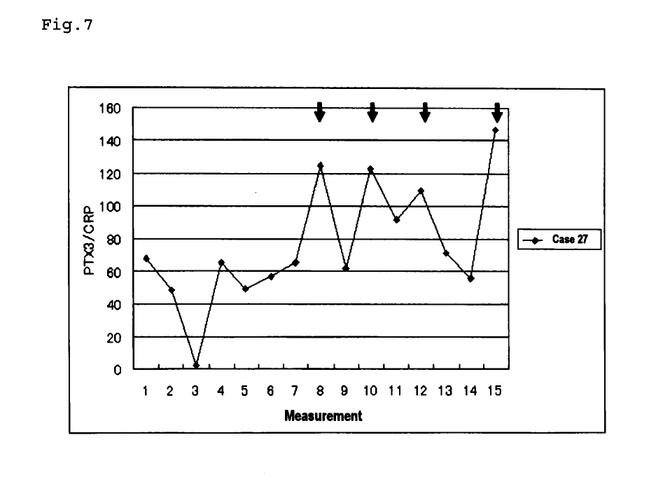

The PTX3/CRP ratios of the two patients described in Example 3 were calculated, and the changes over time in PTX3/CRP ratio are shown in FIG. 6. The change over time in PTX3/CRP of the patient described in Example 4 is shown in FIG. 7. The PTX3/CRP ratio was calculated by using a PTX3 level (ng/mL) and a CRP level (mg/dL). In the case where the measurement of CRP was 0, the CRP level was assumes as 0.1 mg/mL, which is the minimum detection sensitivity of the measurement system.

As a result, the PTX3/CRP ratio was found to be elevated at the time of inflammation caused by aggravation of infection and rheumatoid arthritis (denoted by arrows in FIGS. 6 and 7). When the PTX3/CRP ratio is in excess of about 100, the patient can be suspected to have a certain inflammatory condition.

What is claimed is:

1. A method for assessing an inflammatory condition of a patient who is receiving an IL-6 inhibitor comprising determining a PTX3 level of a sample derived from the patient who is receiving an IL-6 inhibitor.

2. The assessing method according to claim 1, wherein the inflammatory condition is an inflammatory condition attributed to a disease to which an IL-6 inhibitor is applied, or an inflammatory condition attributable to an infection.

3. The assessing method according to claim 1 or 2, wherein the inflammatory condition is assessed by means of the PTX3 level in combination with a CRP level of the sample derived from the patient who is receiving an IL-6 inhibitor.
